(19) United States
(12) Patent Application Publication
(10) Pub. No.: US 2025/0316052 A1
(43) Pub. Date: Oct. 9, 2025

(54) Training Method for Training a Machine Learning Algorithm, Segmentation Method, Computer Program Product and Segmentation Device

(71) Applicant: BASF SE, Ludwigshafen Am Rhein (DE)

(72) Inventors: Phil Jack Holzmeister, Mannheim (DE); Sebastian Michael Fritsch, Ludwigshafen (DE); Maik Friedel, Ludwigshafen (DE)

(21) Appl. No.: 18/864,735

(22) PCT Filed: May 12, 2023
(86) PCT No.: PCT/EP2023/062845

§ 371 Date: Nov. 11, 2024

(30) Foreign Application Priority Data
May 12, 2022 (EP) 22173024.5

(51) Int. Cl. G06V 10/26; G06V 10/82; G06V 20/69; G16C 20/70

(52) U.S. Cl. CPC G06V 10/26; G06V 10/82; G06V 20/69; G16C 20/70

(57) ABSTRACT

A training method for training a machine learning algorithm to perform image segmentation on an image of a chemical substance comprises the steps of: receiving input data including at least partly labeled images of the chemical substance identifying a shape and a position of the chemical substance: receiving a machine learning algorithm framework: training the framework using the input data to obtain a candidate machine learning algorithm for outputting a prediction indicating a shape and position the chemical substance on input images; and calculating a validation metric for the candidate machine learning algorithm, the validation metric being an intersection over union (IoU) per instance)

The present invention relates to a training method for training a machine learning algorithm to perform image segmentation on an image of a chemical substance. The present invention further relates to a segmentation method using the trained machine learning algorithm, a computer program product and a segmentation device.

The segmentation of images representing chemical substances can be useful to determine properties like shape, size and morphology of the chemical substances. Based on the determined properties, the preparation conditions and/or a material performance of the chemical substance can be determined. A quantitative analysis of the above properties from microscopic images often requires very large effort. For example, if a machine learning algorithm is used for segmentation, a large amount of fully annotated data is usually required.

It is one object of the present invention to improve the segmentation of images representing a chemical substance.

In chemical industries improving the technical properties of chemical materials or developing new chemical materials is a key task. For this samples need to be manufactured and analyzed. This requires a high amount of resources in laboratory equipment as well as in analysis equipment. Image based analysis complements other techniques. Image segmentation may be used on the sample image data, allowing to identify chemical substances such as particles and/or different structures in the sample data. Segmentation models require fully labeled images for training. In development of new chemical materials this is often not possible at early stages of development. This may be the case if not all chemical substances in the image data can be identified by their shape and position. The proposed method allows a machine learning algorithm to perform image segmentation on an image of a chemical substance using partially labeled images. This is beneficial, where not all structures in an image can yet be attributed to a label. This allows to perform image segmentation already at an earlier stage in the development process. This speeds up the development process.

Furthermore, fully labeled, e.g. annotated images require substantial efforts from chemical experts. Labeling may lead to errors, in particular, when structures on the images appear ambiguous. The proposed method allows using partially labeled images for training, therefore reducing ambiguity and errors. This leads to a more robust segmentation model.

According to a first aspect, a training method for training a machine learning algorithm to perform image segmentation on an image of a chemical substance is provided. The training method comprises:
receiving input data including at least partly labeled images of the chemical substance identifying a shape and a position of the chemical substance on the at least partly labeled image;
receiving a machine learning algorithm framework having a corresponding set of hyperparameters;
training the machine learning algorithm framework using the input data to obtain a candidate machine learning algorithm for outputting a prediction indicating a shape and position of the chemical substance on input images received as an input; and
calculating a validation metric for the candidate machine learning algorithm, the validation metric including an intersection over union (IoU) per instance, the IoU per instance being a ratio of an overlapping area to a union, the overlapping area being the largest area of overlap between a labeled chemical substance from one of the at least partly labeled images and the prediction by the candidate machine learning algorithm on a corresponding input image corresponding to the one of the at least partly labeled images, and the union being a union of the labeled chemical substance from the one of the at least partly labeled images and the prediction by the candidate machine learning algorithm on the corresponding input image.

The validation metric is indicative of how well the candidate machine learning algorithm performs segmentation, even in the case in which the input data includes non-labeled (unlabeled) regions. Since the input data needs to be only partly labeled, a labeling effort can be reduced. Accordingly, a number of images included in the input data can be increased, thereby advantageously improving the training of the machine learning algorithm. The trained machine learning algorithm can be used to perform segmentation on an image of a chemical substance with reduced effort.

The chemical substance (for example a particle) can be a form of matter having constant chemical composition and characteristic properties. In embodiments the chemical substance includes solid state particles. Further examples of chemical substances include polymers, crystals and molecules. Polymers can be made up a number of joined-together monomers.

The machine learning (ML) algorithm trained according to the training method can be designated as “trained ML algorithm”, “segmentation ML algorithm” and/or “segmentation algorithm”. The trained segmentation ML algorithm allows segmenting an image received as an input into different portions. The segmentation ML algorithm can be a Deep Learning algorithm and/or a neural network algorithm.

“Segmentation” here in particular designates splitting the image into different regions depending on their characteristics. In detail, segmentation can here refer to the identification, for a given region or pixel of the image, whether it shows and/or belongs to the chemical substance or not. The segmentation can be a score (for example between 0 and 1) indicating, for each region or pixel of the image, how likely it is to be part of the chemical substance.

The input data includes at least partly labeled images, which can be considered as training data. The at least partly labeled images can include one or multiple chemical substance labels, wherein the labels indicate a shape and position of the chemical substance in the image. In particular, the label can indicate an outline of the chemical substance, a center of the chemical substance, a geometrical shape approximating the actual shape of the chemical substance and/or the like. The labeling of the chemical substance can be performed manually by a user, for example. In an embodiment, the data including at least partly labeled images comprises at least one partly labeled image. In an embodiment, partly labeled image may refer to an image, where the number of labeled objects is lower than a maximum number of objects in the image. In an embodiment, machine learning algorithm framework may refer to a machine learning model, in particular a convolutional neural network.

The machine learning algorithm framework (or model) can be defined through its corresponding set of hyperparameters, wherein hyperparameters may refer to parameters associated with a property of the machine learning model.

In an embodiment, a machine learning algorithm to perform image segmentation on an image of a chemical substance is provided may be referred to as segmentation model.

In an embodiment, a hyperparameter may refer to a parameter associated with a property of a machine learning model. Property of the machine learning model may refer to a structure of the machine learning model. This may e.g. refer to a number of layers of neural networks, the in particular with a structure, an architecture or a functionality of the machine learning algorithm. The hyperparameters can be parameters or settings that can be modified to adjust a structure, architecture and/or functionality of the machine learning algorithm framework. The set of hyperparameters may include, as hyperparameters, the number of convolutional layers, the dropout (fraction of randomly left out neurons), or the like. The hyperparameters can be selected manually by a user or selected automatically, for example randomly or in a predefined order. Suggesting new hyperparameters (modifying the hyperparameters) can refer to optimizing the model training and can be performed by following one of the following strategies: random search. Bayesian optimization, grid search and the like.

In the training step, the machine learning algorithm framework is trained to obtain a candidate machine learning algorithm. The candidate machine learning algorithm can correspond to the machine learning algorithm framework, to which determined weights (of each layer) have been assigned. The weights can be determined based on the input data. In particular, the training step for obtaining a candidate model is based on the provided set of parameters associated with the structure of the machine learning model. Hence, during the training step, the structure of the machine learning model remains unchanged. The training step may comprise determining weights and/or biases between connected neurons. General concepts of training CNNs are described in “Deep Learning in Neural Networks: An Overview Technical Report” (arXiv: 1404.7828 v4).

The candidate machine learning algorithm can be a candidate for the trained segmentation ML algorithm. The candidate machine learning algorithm can be selected and/or used as the trained segmentation ML algorithm. The candidate machine learning algorithm is capable of receiving, as an input, input images. These input images can include representations of one or multiple chemical substances. At least some of the input images are unlabeled, meaning that not all chemical substances of the input images are labeled. The input images may be fully unlabeled.

The candidate ML algorithm is in particular trained such that it can perform segmentation of the input images and provide the result of this segmentation as an output. The result of this segmentation can be the prediction indicating a shape and position of the chemical substance on the input images input into the candidate ML algorithm. In particular, the prediction is a segmentation of the input image input into the candidate ML algorithm.

The candidate ML algorithm is evaluated by calculating the validation metric. In particular, the validation metric is indicative of how well the candidate ML algorithm performs, in particular of how well it predicts the shape and position of chemical substances in the input images input into the candidate ML algorithm.

The validation metric (validation parameter) can be or include an IoU per instance. The IoU per instance is slightly different from the “normal” or “standard” IoU. Namely, the normal or standard IoU is indicative of the intersection for labels and predictions across the full image. The normal or standard IoU can be the number X of pixels in the intersection divided by the number Y of pixels in the union.

The IoU per instance evaluates this for each labeled region separately and only with the largest overlapping predicted region. In other words, for each ground-truth-instance (for each at least partly labeled image), the predicted labeled region with the largest overlap (Omax) with the ground-truth-instance (G) is determined. The IoU per instance corresponds to the area of the overlap (Omax, G) divided by the area of the union (Omax, G) for respective instances. The use of the proposed validation metric related to the IoU per instance allows the use of partly labeled images. This is realized by ignoring predicted shapes and positions of the chemical substance in unlabeled areas of the image. Which may lead to Hence, allowing to perform image segmentation already at an earlier stage in the development process. This speeds up the development process.

In detail, for each at least partly labeled image, a corresponding input image can be retrieved or generated. In particular, the corresponding input image includes the same representation of the chemical substance as the corresponding at least partly labeled image, but without any or without some of the labels. The corresponding input image can be used to generate the corresponding at least partly labeled image through labeling. For example, the corresponding input image may be the unlabeled version of the at least partly labeled image corresponding thereto. The corresponding input image may include the same representation of chemical substances as the corresponding at least partly labeled image, but without the labels or with less or more labels. The candidate ML algorithm can be applied onto the corresponding input image to obtain a prediction of the shape and position of the chemical substances. The area of each prediction, namely the area of each predicted chemical substance, can be calculated. The area (size) can correspond to the number of pixels in the predicted chemical substance.

In each at least partly labeled images, the area (size) of the labeled regions may be calculated, for example as the number of pixels. For an at least partly labeled image and the corresponding input image, the area of the intersection (overlapping area) can be determined by calculating how many pixels intersect between the predicted chemical substance and the corresponding labeled chemical substance from the input data, for a given identified chemical substance (region). The largest overlapping area can be determined by comparing the number of pixels of each intersection. For the at least partly labeled image and the corresponding input image, the area of the union (joint area) can be determined by calculating a union of all pixels belonging to the predicted chemical substance and the corresponding labeled chemical substance from the input data for a given identified chemical substance (region). The IoU per instance (per image) may be determined by calculating the ratio between the area of intersection and the area of union for the largest overlapping area.

As compared with the normal or standard IoU, with the IoU per instance, predictions in unlabeled regions (of the partly labeled images) do not negatively affect the validation metric.

Thus, the validation metric is indicative of how well the candidate ML algorithm performs segmentation, even if the input data includes non-labeled (unlabeled) regions. Since the input data needs to be only partly labeled, a labeling effort can be reduced. Accordingly, a number of images included in the input data can be increased, thereby advantageously improving the training of the ML algorithm.

Disclosed according to a further aspect is a training method for training a segmentation model to perform image segmentation on an image of a chemical substance, the training method comprising:
receiving input data including at least partly labeled images comprising one or more partly labeled images of the chemical substance, wherein the label comprises a shape, in particular a set of pixels associated a chemical substance in the image and a position of the chemical substance on the at least partly labeled images;
receiving a machine learning model, in particular comprising a convolutional neural network, having a corresponding set of parameters associated with a structure of the machine learning model;
training the machine learning algorithm model using the input data to obtain a candidate segmentation model for outputting a prediction indicating a shape and position of the chemical substance on input images received as an input, while maintaining the provided set of parameters; and
calculating a validation metric for the candidate machine learning algorithm, the validation metric including an intersection over union (IoU) per instance, the IoU per instance being a ratio of an overlapping area to a union per instance instance, the overlapping area being the largest area of overlap between a labeled chemical substance from one of the at least partly labeled images and the prediction by the candidate machine learning algorithm on a corresponding input image corresponding to the one of the at least partly labeled images, and the union being a union of the labeled chemical substance from the one of the at least partly labeled images and the prediction by the candidate machine learning algorithm on the corresponding input image.

In an embodiment, the training method according to claim 1, further comprising, based on the value of the calculated validation metric, in a new iteration:
Providing a set of parameters associated with a structure of the segmentation model, different from the set of parameters initially provided, thereby amending the structure of the segmentation model; and
repeating the steps of receiving the machine learning model, training the machine learning model and calculating the validation metric for the different set of parameters associated with a structure of the machine learning model.

In an embodiment the training method further comprises:
storing and/or outputting a current candidate machine learning algorithm as a trained machine learning algorithm for performing image segmentation if the calculated validation metric is determined as being greater than or equal to a predetermined validation threshold; and/or
storing and/or outputting the candidate machine learning algorithm with the highest validation metric amongst candidate machine learning algorithms from multiple iterations.

In an embodiment, the training method disclosed herein, wherein the segmentation model is configured to take an image, perform a learned transformation of the image, wherein performing the learned transformation refers to segmentation, and output a list of shapes in the image; wherein the list of shapes refers to shapes identified in the image wherein the machine learning algorithm has a free parameter, in particular a weight that is optimized by heuristic optimization during the training.

In an embodiment the training method disclosed herein, wherein the machine learning model is one of the following machine learning models: U-Net or Mask-RCNN (region based convolutional neural network).

In an embodiment, the training method disclosed herein, wherein the chemical substance is a particle made of a cathode active material, nickel, cobalt and/or manganese.

In an embodiment, the training method disclosed herein, wherein the at least partly labeled images of the chemical substance are scanning electron microscope (SEM) images.

In an embodiment the training method disclosed herein, further including calculating IoU scores using multiple values of an IoU metric and determining a selected value of the IoU metric, the selected value of the IoU metric being the value out of the multiple values of the IoU metric leading to the highest IoU score.

In an embodiment the training method disclosed herein, wherein the validation metric corresponds to the IoU per instance score calculated with the selected value of the IoU metric.

The segmentation method of claim 11, further comprising
determining (S14) a technical performance parameter value of the chemical substance (3) using a performance model, wherein the performance model is parametrized based on technical performance values and features of the chemical substance and using the determined features of the chemical substance (3) as an input to the performance model
providing the technical performance property.
13. The segmentation method according to claim 12, wherein
   the performance model is a machine learning model trained using performance training data including features of chemical substances (3) and corresponding performances, the trained performance model being configured to take the features of the represented chemical substance (3) determined through image analysis as an input and to provide performance parameter values as an output.

According to an embodiment, the training method further comprises, based on the value of the calculated validation metric, (in a new iteration):
providing a different set of hyperparameters; and
repeating the steps of receiving the machine learning algorithm framework, training the machine learning algorithm framework and calculating the validation metric for the different set of hyperparameters.

The different set of hyperparameters may be modified by a user or updated automatically, in particular taking into account the value of the calculated validation metric. The provision of new hyperparameters (modifying the hyperparameters) can refer to optimizing the model training and can be performed by following one of the following strategies: random search, Bayesian optimization, grid search and the like.

In an embodiment, the training method further comprises based on the value of the calculated validation metric varying the set of parameters associated with the structure of the machine learning model. This may lead to a set of different parameters associated with the structure of the machine learning model. This may be understood as changing the structure of the machine learning model. Examples may be an increase or decrease of a kernel size, and increase or decrease of the convolutional layers.

Using the machine learning algorithm framework having the different (new) set of hyperparameters, the steps of receiving the machine learning algorithm framework, training the machine learning algorithm framework and calculating the validation metric can be performed for the different set of hyperparameters. The repetition of these steps can correspond to a new iteration of the training process. Performing multiple iterations of the training process can allow improving and/or optimizing the candidate machine learning algorithm. At each iteration, the calculation of the validation metric in particular allows evaluating the current candidate ML algorithm (of the current iteration) and deciding whether or not to continue the training and/or how to modify the set of hyperparameters.

According to a further embodiment, the training method further comprises:
storing and/or outputting a current candidate machine learning algorithm as a trained machine learning algorithm for performing image segmentation if the calculated validation metric is determined as being greater than or equal to a predetermined validation threshold; and/or
storing and/or outputting the candidate machine learning algorithm with the highest validation metric amongst candidate machine learning algorithms from multiple iterations.

For example, the training method may stop as soon as the validation metric reaches a predetermined validation threshold. In this case, the current candidate ML algorithm (which lead to this good validation metric) can be kept as the (final) trained ML algorithm. The (final) trained ML algorithm can be stored and/or output to be later used in image segmentation.

The training method may be performed until a predetermined number of training iterations are reached and/or until a validation metric stagnates. In this case, the training method can be stopped and the candidate ML algorithm leading to the highest validation metric can be kept as the (final) trained ML algorithm. The (final) trained ML algorithm can be stored and/or output to be later used in image segmentation.

According to a further embodiment, the machine learning algorithm framework is a machine learning model configured to take an image (any input image or test image), perform a learned transformation of the image, and output a list of shapes in the image; wherein the machine learning algorithm has a free parameter that is optimized by heuristic optimization during the training.

In particular, by “shapes”, a set of pixels describing an object (chemical substance) in the image is here meant. The free parameter can be the weights. The heuristic optimization can be a stochastic gradient descent. The heuristic optimization may be performed to be able to produce the desired output (list of shapes) on provided training data.

According to a further embodiment, the machine learning algorithm framework is one of the following machine learning models: U-Net or Mask-RCNN (region based convolutional neural network).

U-Net and Mask-RCNN mostly differ in the exact way the learned transformation of an input is done, but the remaining training procedure is comparable.

In the case of U-Net, the hyperparameters tuned during training can include the dropout (fraction of randomly left out neurons), the number of convolutional layers (depth), weights of the pixel-wise loss (e.g. higher weight for pixels separating two instances) and/or the like. For Mask-CNN, the hyperparameters tuned during training include weights of the loss function (class, box, mask) and the like.

According to a further embodiment, the chemical substance is a particle made of a cathode active material, nickel, cobalt and/or manganese.

According to a further embodiment, the at least partly labeled images of the chemical substance are scanning electron microscope (SEM) images.

According to a further embodiment, the method further includes calculating IoU scores using multiple values of an IoU metric and determining a selected value of the IoU metric, the selected value of the IoU metric being the value out of the multiple values of the IoU metric leading to the highest IoU score.

In particular, the outputs of U-Net and Mask-RCNN include scores (such as a value between 0 and 1 that describes how likely a pixel/object is a chemical substance). A metric is used which is not bound to a fixed score level, but instead always measures the “best” intersection over union that can be achieved by tuning the score threshold. This metric is the IoU metric (IoU per instance metric). The metric defines a list of score thresholds to try, for example [0.1, 0.2, . . . 0.9].

An example for calculating IoU per instance scores is as follows:
1) For each score threshold t (i.e. for each value of the IoU metric): calculate Sum (IoU per instance)/N_labels, wherein Sum (IoU per instance) is the sum of all IoU per instance over all N_images images, N_labels is the number of labels. In a further embodiment, IoU per instances smaller than an IoU threshold t2 may be left out of the sum. In an embodiment, e.g. IoU per instances smaller than t2=0.5 are left out of the sum.
2) The selected and used final IoU per instance metric (i.e. the selected value of the IoU metric) is max_t IoU (t), namely the score threshold (the value of the IoU metric) for which the IoU per instance has its maximum.

One may contemplate alternative IoU metrics. For example, an average of max_t IoU (t) with respect to all images N_images can be used and stored as selected IoU per instance.

The selected IoU per instance metric can be stored and used to improve the calculation and/or reliability of the validation metric. In particular, the validation metric can be calculated as a function of the IoU per instance and the selected IoU per instance metric. The validation metric can correspond to the IoU per instance score calculated with the selected value of the IoU metric and be calculated along the lines above.

According to a further embodiment the calculation of the IoU scores includes, for each value of the IoU metric, calculating a sum of all IoU per instances divided by the number of labels, wherein IoU per instances smaller than the value of the IoU metric are left out of the sum.

According to a further embodiment, the validation metric corresponds to the IoU per instance score calculated with the selected value of the IoU metric.

In embodiments the method for training a machine learning algorithm is implemented as a software service, in particular in terms of a software as a service (SaS) in a cloud environment, e.g. Amazon web service (AWS), Microsoft Azure, Siemens Mindsphere or the like.

One aspect includes the trained machine learning algorithm, i.e. the machine learning algorithm trained according to the above or below training method.

According to a second aspect, a segmentation method for performing segmentation of data representing a chemical substance using a trained machine learning algorithm trained according to the training method of the first aspect or according to any embodiment thereof is provided. The segmentation method includes:
receiving at least partially unlabeled data to be segmented, the at least partially unlabeled data including an image of the chemical substance;
inputting the at least partially unlabeled data into the trained machine learning algorithm; and
outputting, by the trained machine learning algorithm, label data indicating a shape and position of the chemical substance on the image of the chemical substance.

The segmentation method allows using the trained machine learning algorithm described above for segmentation purposes, in particular to identify locations (positions) and shapes of chemical substances. The trained ML algorithm can receive at least partly unlabeled data to be segmented for segmentation. The at least partly unlabeled data can be SEM images. The at least partly unlabeled data can be entirely unlabeled or partly labeled, wherein the trained ML algorithm is used to find the labels of the remaining shown chemical substances.

The output of the trained ML algorithm can be the label data. In particular, the label data can be indicative where chemical substances are located in the images, in particular their positions and shapes. The label data can be superimposed on the image or provided as metadata and/or data characterizing the image. The label data can be indicative, for each pixel, how likely it is to belong to a chemical substance.

According to an embodiment, the segmentation method further includes:
using the label data, performing an image analysis to determine features of the represented chemical substance.

It is understood that the term input data may refer to test, training and data to be analysed/evaluated through a segmentation process throughout this disclosure.

The image analysis can be performed using a conventional image analysis tool. Features of the chemical substance include size parameters (an area, surface, diameter, length, perimeter, smallest convex area around the chemical substance, radius of largest circle inside the chemical substance or the like), shape parameters (dimensionless shape descriptions regardless of the absolute size of the chemical substance, for example compactness, sphericity, aspect ratio, form factor or the like), intensity based parameters (derived from pixel intensities, such as a median intensity, standard deviation of intensity of all pixels in a region, and the like), a number of holes in a region, a number of edges in a region, an identifier of the region, a coordinate of the center of mass of the region and/or the like.

According to a further embodiment, the segmentation method further comprises at least one of the following:
determining a technical performance value of the chemical substance using a performance model and using the determined features of the chemical substances as an input to the performance model; and/or
predicting particle properties using a particle prediction model and using the determined features of the chemical substances as an input to the particle prediction model.

According to a further embodiment,
the performance model is a machine learning model trained using performance training data including features of chemical substances and corresponding performances, the trained performance model being configured to take the features of the represented chemical substance determined through image analysis as an input and to provide electrochemical performance parameters as an output; and/or
the particle prediction model is a machine learning model trained using prediction training data including process conditions of interest, corresponding particle features, the trained particle prediction model being configured to take the features of the represented chemical substance determined through image analysis and process conditions of interest as an input and to provide processing conditions to generate a desired particle as an output.

Assuming the goal of process development is to produce spherical chemical substances (particles) of homogeneous target size. For this, one can would train a ML model (particle prediction model) on a relevant dataset (e.g. from initial experiments, or subset from larger database), which takes the process conditions of interest (e.g. temperature, stirring speed, concentrations, atmosphere, duration of process steps and the like) as input, and the average sphericity (formfactor in the list above) as well as the average and standard deviation of the particle size as output. Based on these models, processing conditions can be suggested that should generate the desired particle features.

Further, one can train a ML model (performance model) which takes the features produced from the image analysis (depending on the scope together with material composition and process conditions) as input; and the electrochemical performance metrics (e.g. capacity or long term stability) as an output. From this, it can be seen if and/or which particle properties effect the performance. Some of the parameters that typically influence performance are the particle size distributions, the particle surface area, but it can also be things like the orientation of primary crystals (homogenously radial from particle center or disordered).

According to a further aspect a method for estimating a performance of a material or substance is presented, wherein the method comprises the steps of:
receiving an image of the material or substance;
segmenting the image using a first trained machine learning algorithm, said first trained machine learning algorithm outputting data indicating a shape and position of the material or substance; and
estimating a performance of the material or substance using a second trained machine learning algorithm, said second trained machine learning algorithm receiving data indicating a shape and position of the material or substance and outputting a performance parameter of the material or substance.

In embodiments, the first machine learning algorithm is trained according to the above or below with respect to embodiments of the segmentation method, the second machine learning algorithm is trained according to the above or below with respect to embodiments of the performance model, and/or the performance parameter is output as a parameter of interest of the material or substance in relation to a chemical production process for producing the material or substance.

According to another aspect a method for predicting a property or characteristic of a material or substance is presented, wherein the method comprises the steps of:
receiving process conditions for manufacturing the material or substance;
predicting a property or characteristic of the material or substance as a function of the process conditions using a third trained machine learning algorithm.

In embodiments, the third trained machine learning algorithm further receives data indicating a shape and position of the material or substance output from the first trained machine learning algorithm, the third machine learning algorithm is trained according to the above or below with respect to embodiments of the particle prediction model, and/or the property or characteristic of a material or substance is output as a function of the received process conditions.

In embodiments the method for predicting a property or characteristic of a material or substance comprises at least one of the steps of:
setting a target property; and
varying the process conditions;
comparing the predicted property or characteristic of the material or substance to the target property to obtain a similarity measure.

The process conditions may be varied until the similarity measure is below a predetermined threshold. A similarity measure can be a norm, distance, difference or the like depending on a parametrization of the target property. The target property may be parametrized by a multidimensional vector. A target property is, for example a hardness, a porosity, an electrical resistance, etc.

The material or substance consists of solid-state particles in embodiments.

It is an advantage that either a single or few images of the material or substance is/are required to estimate its performance under various process conditions instead of conducting experiments. Process conditions are, in particular, taken into account through the respective training processes of the employed machine learning algorithms. Hence, less resources in terms of laboratory capacities and shipping test samples are required.

The methods allow for a validation of process conditions in manufacturing processes for the respective materials or substances prior to implementing those in the field. Further, the methods allow for predicting characteristics of hitherto not manufactured materials or substances. Hence, development and research of new materials is improved.

The embodiments and features described with reference to the training method of the first aspect or an embodiment thereof apply mutatis mutandis to the segmentation method of the second aspect.

According to a third aspect, a computer program product comprising instructions which, when the program is executed by a computer, cause the computer to carry out the method according to the first or second aspect or any embodiment thereof.

A computer program product, such as a computer program means, may be embodied as a memory card, USB stick, CD-ROM, DVD or as a file which may be downloaded from a server in a network. For example, such a file may be provided by transferring the file comprising the computer program product from a wireless communication network.

The embodiments and features described with reference to the methods of the first and second aspects and any embodiments thereof apply mutatis mutandis to the computer program product of the third aspect.

According to a fourth aspect, a segmentation device is provided. The segmentation device includes:
a storage unit for storing a trained machine learning algorithm trained according to the method according to the first or second aspect or according to any embodiment thereof;
an input unit for receiving at least partially unlabeled data to be segmented, the at least partially unlabeled data including an image of the chemical substance;
a processor configured to input the at least partially unlabeled data into the trained machine learning algorithm to determine label data indicating a shape and position of the chemical substance on the image of the chemical substance; and
an output unit for outputting the determined label data.

The segmentation device can be part of a computer, in particular of a personal computer or of an industrial computer. The storage unit storing the trained ML algorithm may be any type of temporal or permanent storage (memory). The processor may be a central processing unit (CPU) or the like which is configured to execute the trained ML algorithm stored therein and/or to perform the methods according to the first or second aspects or according to any embodiment thereof. The input unit can include a user interface to receive the at least partly unlabeled data from the user, or it can be a unit that directly receives the at least partly unlabeled data from an SEM or the like. The output unit can be a user interface, such as a display, touchscreen, or the like.

The embodiments and features described with reference to the methods of the first and second aspects and any embodiments thereof apply mutatis mutandis to the segmentation device of the fourth aspect.

Further possible implementations or alternative solutions of the invention also encompass combinations—that are not explicitly mentioned herein—of features described above or below with regard to the embodiments. The person skilled in the art may also add individual or isolated aspects and features to the most basic form of the invention. It is understood, that the methods can be executed as computer-implemented methods.

Further embodiments, features and advantages of the present invention will become apparent from the subsequent description and dependent claims, taken in conjunction with the accompanying drawings, in which:

FIG. 1 shows a training method according to a first embodiment;

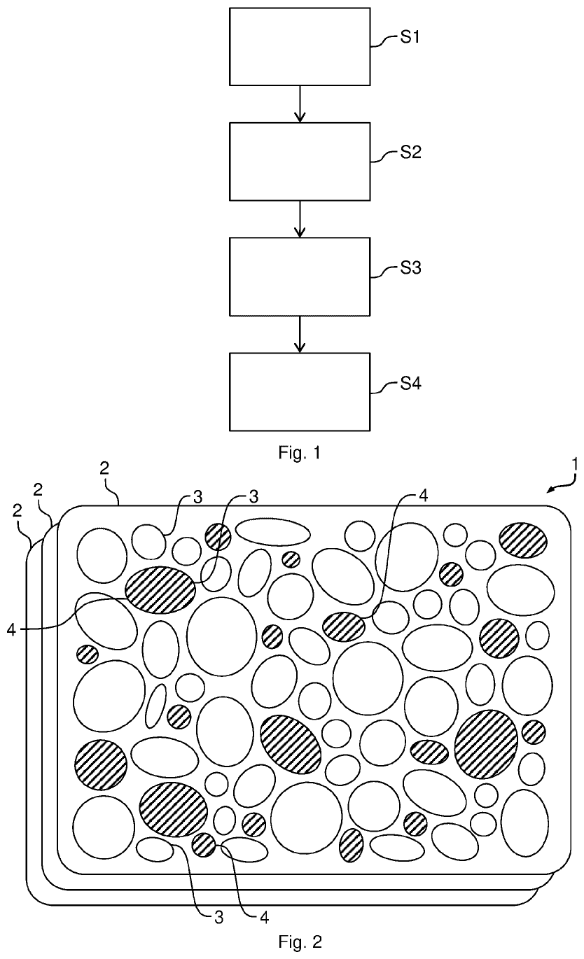

FIG. 2 shows an example of input data;

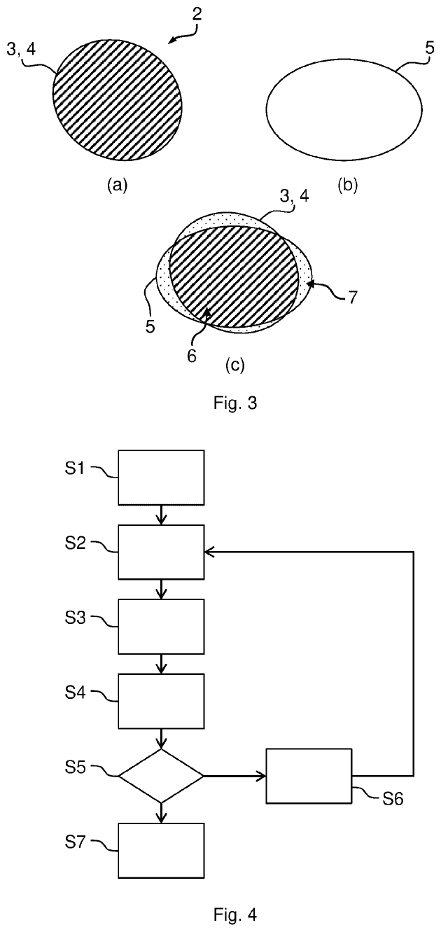

FIG. 3 shows examples of (a) a labeled chemical substance, (b) a predicted chemical substance, and (c) an overlap and union;

FIG. 4 shows a training method according to a second embodiment;

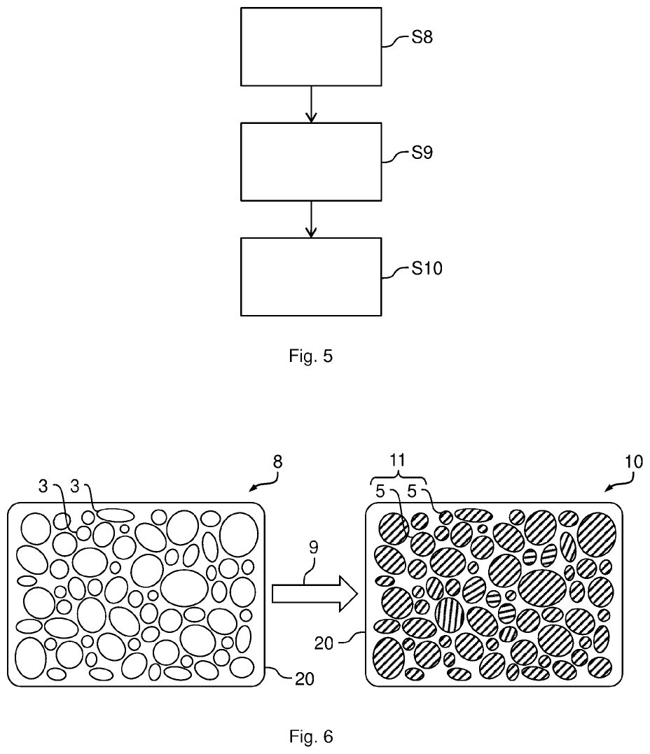

FIG. 5 shows a segmentation method according to a first embodiment;

FIG. 6 shows an example of a segmentation;

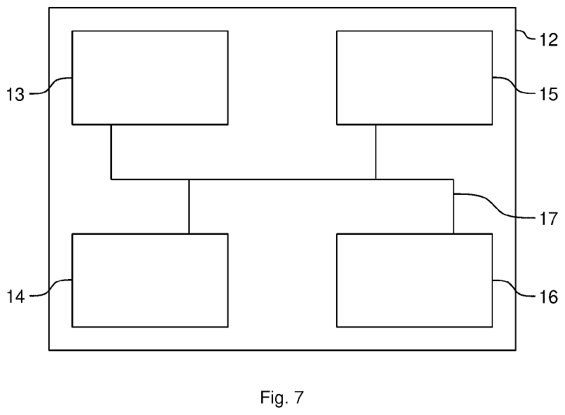

FIG. 7 shows a segmentation device according to an embodiment;

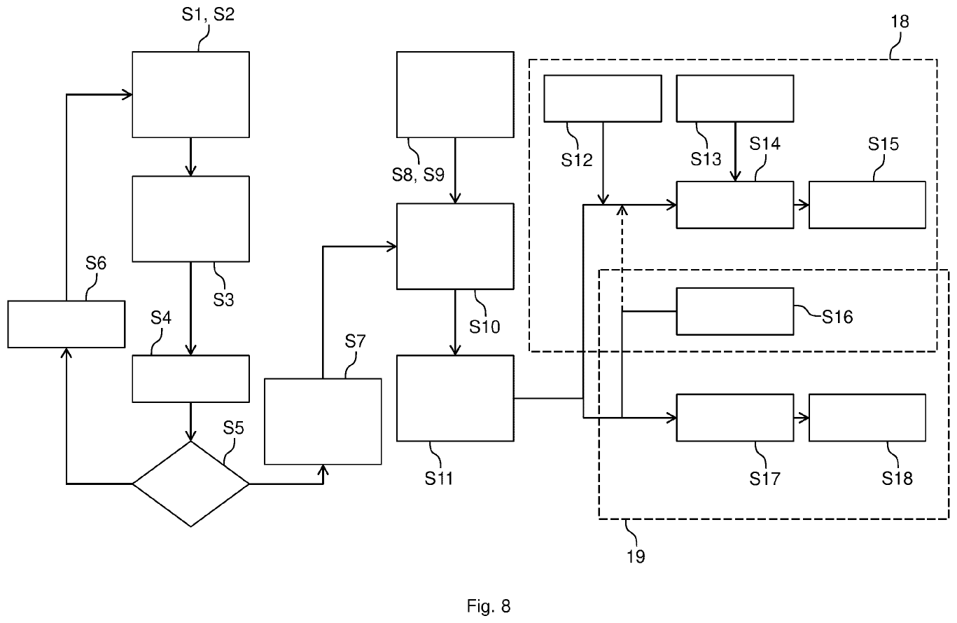

FIG. 8 shows a segmentation method according to a second embodiment; and

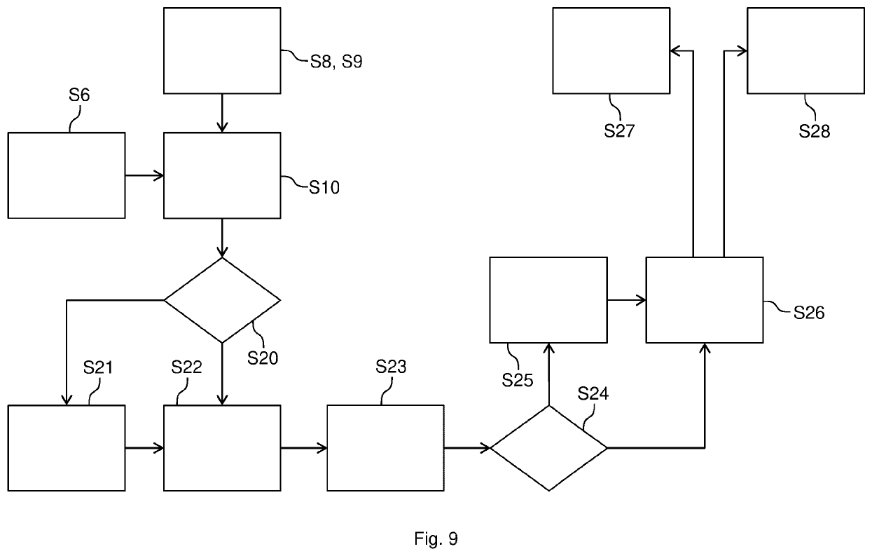

FIG. 9 shows a portion of a segmentation method according to a third embodiment.

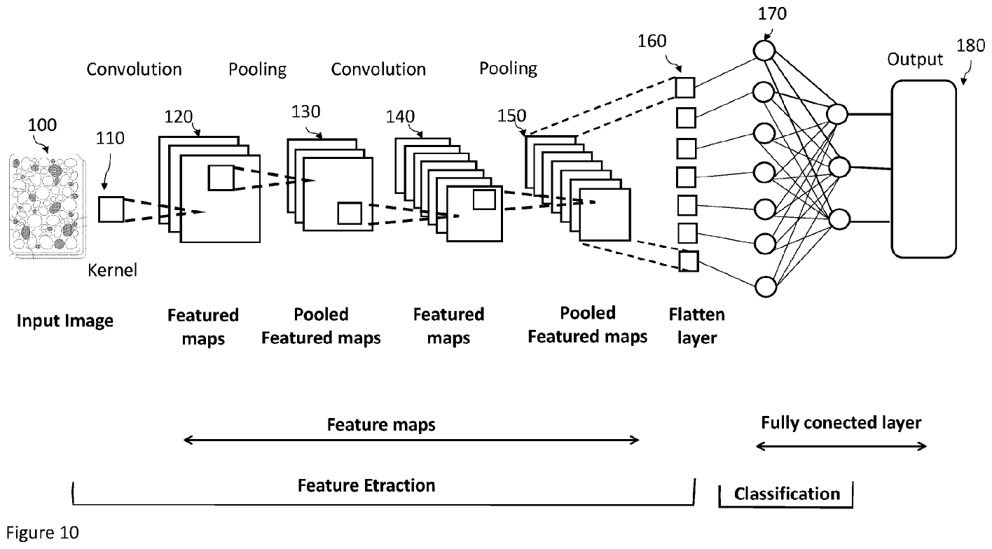

FIG. 10 shows an example a CNN.

In the Figures, like reference numerals designate like or functionally equivalent elements, unless otherwise indicated.

FIG. 1 shows a training method according to a first embodiment. The shown training method is for training a machine learning algorithm to perform image segmentation on an image of a chemical substance. The training method can be performed by a processor of a computing system.

In a step S1 of FIG. 1, input data 1 is received. An example of such input data 1 is shown in FIG. 2. The input data 1 may include multiple images 2, which are at least partly labeled images of chemical substances 3, which are here particles. In detail, the images 2 are scanning electron microscope (SEM) images showing multiple chemical substances 3. Some of these chemical substances 3, but not all, have been labeled by a user. The labeled chemical substances 3 have the reference numeral “4” and represented in a dashed manner. The labels are indicative of the position and shape of the labeled chemical substances 4. The labels provide a visual indicator for the pixels of the images 2 representing chemical substances 3 and those representing something else, such as background. In the example of FIG. 2, three images 2 are shown as belonging to the input data 1, but it is understood that the input data 1 may actually includes many more images 2. Further, in FIG. 2, only some of the chemical substances 3 have reference signs but it is understood that all shown shapes of the images 2 represent chemical substances 3 of the same type. Type may refer to a particle with identical chemical properties.

In a step S2, a machine learning algorithm framework is received. The machine learning algorithm framework is characterized by a corresponding set of hyperparameters. In the present example, the machine learning algorithm framework and the corresponding set of hyperparameters is selected by a user. In alternative embodiments, they can be automatically selected in accordance with some selection rules.

Here, the machine learning algorithm framework is a U-Net algorithm. The hyperparameters define the dropout (fraction of randomly left out neurons), the number of convolutional layers (depth), and the weights of the pixel-wise loss (e.g. higher weight for pixels separating two instances). The selection of a U-Net algorithm rather than another type of algorithm (such as Mask-RCNN) can also be defined in a hyperparameter.

The received machine learning algorithm framework is a framework to be trained based on the input data 1 (training data) in order to obtain a trained ML algorithm capable of performing segmentation of any input (input image or test image) showing chemical substances 3.

In a step S3 of the training method of FIG. 1, the ML algorithm framework is trained using the input data 1 to obtain a candidate ML algorithm. The candidate ML algorithm is trained such that, when it receives input images representing the chemical substance 3 as an input, it outputs a prediction indicating a shape and position of the chemical substance 3 in the input images. In other word, the candidate ML algorithm is capable of performing a segmentation of the input images representing the chemical substance 3, which corresponds to the performed prediction.

In a step S4, the quality and reliability of the candidate ML algorithm is calculated as a validation metric. The validation metric may comprise an intersection over union (IoU) per instance, the IoU per instance being a ratio of an overlapping area to a union for each instance.

The IoU per instance is explained with reference to FIG. 3, which shows examples of (a) a labeled chemical substance 4, (b) a predicted chemical substance 5, and (c) an overlap (intersection) 6 and a union 7. In detail, FIG. 3 (a) corresponds to one of the labeled chemical substances 4 of one image 2 of the input data 1. FIG. 3 (b) shows a prediction of a chemical substance 5 that the candidate ML algorithm performed on a corresponding input image that shows the same as the image 2 of the input data 1 including the label 4, but without the label 4. Thus, FIGS. 3 (a) and 3 (b) show a true (actual) label 4 and a predicted label 5 of a same chemical substance 3, respectively.

FIG. 3 (c) shows the actual label 4 and the predicted label 5 represented one above the other. The intersection (overlap) 6 between the actual label 4 and the predicted label 5 is filed by dashed lines. The union 7 between the actual label 4 and the predicted label 5 corresponds to the intersection 6 and all areas of the substances 4 or 5 which are not in the intersection 6. These areas are represented by dots. The union 7 hence corresponds to a sum of the dashed and dotted portions of FIG. 3 (c). The overlap 6 and the union 7 are expressed as a number of pixels included in the overlap 6 and union 7, respectively.

The region of the image 2 used to calculate the IoU per instance is the region including the chemical substance 3 with the largest overlap 6 between the label 4 and the prediction 5. The IoU per instance is calculated for the chemical substance 3 with the largest overlap 6 by dividing the overlap 6 by the union 7 (i.e. by respectively dividing the number of pixels).

In other words, the IoU per instance is a ratio of an overlapping area 6 to a union 7. The overlapping area 6 is the largest area of overlap between a labeled chemical substance 4 from one of the at least partly labeled images 2 and the prediction 5 by the candidate machine learning algorithm on a corresponding input image corresponding to the one of the at least partly labeled images 2. The union 7 is a union of the labeled chemical substance 4 from the one of the at least partly labeled images 2 and the prediction 5 by the candidate machine learning algorithm on the corresponding input image.

The higher the IoU per instance, the better the candidate ML algorithm. By calculating an IoU per instance as the validation metric, predictions 5 in unlabeled regions of the at least partly labeled images 2 do not negatively affect the validation metric. The training of the ML algorithm can hence be performed on less input data 1 (less images 2) and with less labels 4. An effort for training the ML algorithm can hence be reduced.

FIG. 4 shows a training method according to a second embodiment. The training method of FIG. 4 includes the method steps S1-S4 described in view of FIG. 1. Steps S1-S4 of FIG. 4 corresponding to those of FIG. 1, they will not be described again in the following. In addition to steps S1-S4, FIG. 4 further includes method steps S5-S7.

In a step S5, the validation metric calculated in step S4 is compared with a predetermined validation threshold. If the validation metric is determined as being equal to or greater than the predetermined validation threshold, the current candidate ML algorithm is considered as being performant and is kept as the final trained ML algorithm for segmentation. In this case, in step S7, the current candidate ML algorithm is stored as the final trained ML algorithm in a database.

If in step S5, it is determined that the validation metric is smaller than the predetermined validation threshold, the current candidate ML algorithm is considered as not being sufficiently performant. In this case, another iteration of the training is performed. In detail, in a step S6, a different set of hyperparameters is provided for the machine learning algorithm framework (meaning that a different machine learning algorithm framework is provided). This different set of parameters associated with the structure of the machine learning model may be generated e.g. by random search.

This different set of hyperparameters is selected by a user based on the calculated validation metric. Then, using the different set of hyperparameters and hence the different ML algorithm framework, steps S2-S5 are repeated. In detail, steps S2-S6 are repeated until in step S5, the current validation metric is equal to or higher than the predetermined validation threshold. In this case, the current candidate ML algorithm is kept and stored at step S7, as explained above.

As an alternative or in addition to the selection process of step S5 of FIG. 4, the training method may be performed for a predetermined number of iterations (for example for ten iterations). At the end of the ten iterations, the candidate ML algorithm leading to the highest validation metric is kept. It is also possible to stop the training method either when the predetermined number of iterations or when the current validation metric is equal to or higher than the predetermined validation threshold (whichever happens first).

Repeating the steps S2-S6 allows optimizing the ML algorithm further and obtaining more reliable segmentation results when using the trained ML algorithm.

The trained ML algorithm (the algorithm stored in step S7) can be used to perform segmentation on any images from the SEM. This is performed in accordance with a segmentation method, an example of which is shown in FIG. 5. The method steps S8-S10 of FIG. 5 can be performed after the steps S1-S4 of FIG. 1 or after steps S1-S7 of FIG. 4.

In step S8 of FIG. 5, at least partly unlabeled data 8 to be segmented is received. As shown in FIG. 6, the at least partly unlabeled data 8 includes one or multiple SEM images 20 of a chemical substance 3. In the example of FIG. 6, the at least partly unlabeled data 8 is entirely unlabeled data in which none of the chemical substances 3 is labeled. Alternatively, some but not all of the chemical substances 3 in the data 8 can be labeled. The purpose of the segmentation method is to segment the data 8, namely to identify position and shapes of chemical substances 3 using the trained ML algorithm.

In step S9 of FIG. 5, the at least partly unlabeled data 8 is input into the trained machine learning algorithm. The trained machine learning algorithm is represented by an arrow 9 in FIG. 6.

In step S10 of FIG. 5, the trained ML algorithm 9 outputs or stores label data 11 indicating a shape and position of the chemical substance 3 on the image 20 of the chemical substance 3. In detail, the label data 11 includes predictions 5 of the trained ML algorithm 9 about the position and shapes of the chemical substances 3 in the images 20, as indicated by the dashed chemical substances 3 in the right part of FIG. 6. The images 20 output by the trained ML algorithm form fully labeled data 10 in which all chemical substances 3 are labeled.

It is indicated that in FIG. 6, only some of the chemical substances 3 and some of the predictions 5 have reference signs but it is understood that all shown shapes of the images 20 represent chemical substances 3 and that all striped shapes of the images 20 represent predictions 5.

The segmentation method of FIG. 5 can be performed using a segmentation device 12 shown in FIG. 7. In detail, the segmentation device 12 includes a storage unit 13, an input unit 14, a processor 15 and an output unit 16 all connected via connection cables 17. The storage unit 13 is a random-access memory (RAM) for storing the trained machine learning algorithm 9 trained according to the training method of FIG. 1 or 4.

The input unit 14 is a communication interface which receives the at least partially unlabeled data 8 to be segmented directly from the SEM. The processor 15 is configured to input the at least partially unlabeled data 8 into the trained machine learning algorithm 9 to determine the label data 11 indicating a shape and position of the chemical substance 3 on the image 20 of the chemical substance 20. The processor 15 can be configured to execute the methods of FIGS. 1, 4 and/or 5. The output unit 16 is configured to output the determined label data 16 on a screen and/or to store the label data 16 for later uses.

FIG. 8 shows a segmentation method according to a second embodiment. The segmentation method of FIG. 8 includes steps S1-S10 already described above. Steps S1-S10 are hence not described again. In addition to steps S1-S10, FIG. 8 includes steps S11-S18 which will be described in the following.

The steps S11-S18 are method steps performed using the result of the segmentation by the trained ML algorithm, in particular using the label data 11. In detail, in step S11, an image analysis is performed using an image analysis tool to determine features of the chemical substance 3. The image analysis is performed using the label data 11 indicating position and shapes of the chemical substance 3. The determined features of the chemical substance include size parameters (an area, surface, diameter, length, perimeter, smallest convex area around the chemical substance, radius of largest circle inside the chemical substance or the like), shape parameters (dimensionless shape descriptions regardless of the absolute size of the chemical substance, for example compactness, form factor or the like), intensity based parameters (derived from pixel intensities, such as a median density, standard deviation of intensity of all pixels in a region, and the like), a number of holes in a region, a number of edges in a region, an identifier of the region, a coordinate of the center of mass of the region and/or the like.

Using the features determined in steps S11, two different processes may be performed, which are indicated as a first example 18 and a second example 19 in FIG. 8. It is possible to perform one or both of the examples 18, 19 after step S11 of FIG. 8.

The first example 18 relates to the estimation of performance using a performance model. In detail, the features determined in step S11, performance data (provided at step S12) and the at least partly unlabeled data 8 (provided at step S13) are input into the performance model in step S14. The performance model is a trained ML model. The performance data is data indicative of a scope of the performance prediction, and for example indicates desired particle features such as a particle size distribution, a particle surface area, an orientation of primary crystals or the like. An output of the performance model is shown in step S15 and corresponds to an electrochemical performance parameter (metric) such as the capacity or long term stability of the particle 3.

The second example 19 relates to particle properties prediction using a particle prediction model. In detail, the features determined in step S11 or process information or conditions (provided at step S16) are input into the particle prediction model in step S17. Optionally, the process information or conditions (provided at step S16) are also input into the previously described performance model (step S14).

The particle prediction model is a trained ML model. The process information is data about a process condition of interest (categories for which a value is desired, such as temperature, stirring speed, concentrations, atmosphere, duration and the like) which is planned to be applied onto the particle 3, but has not yet been applied (no SEM images are available) and further includes desired properties of the processed particle 3. An output of the particle prediction model is shown in step S18 and corresponds to processing conditions (values for the temperature, stirring speed, concentrations, atmosphere, duration and the like) that achieve the desired particle features. The output processing conditions allow to synthesize a particle having a desired property thereby reducing the need for prior experiments or test runs of a chemical plant.

FIG. 9 shows a portion of a segmentation method according to a third embodiment. In detail, FIG. 9 shows features S6, S8-S10 previously described as well as steps S20-S28, which can be included in step S11 shown in FIG. 8 and relating to the image analysis to determine features of the chemical substance 3.

In detail, in step S20, it is determined whether postprocessing on the raw output of the segmentation (label data 11) is required or not. If postprocessing is required, it is performed in step S21. Postprocessing can include segmentation of U-Net pixel-score. If no postprocessing is required or after the postprocessing of step S21, in step S22, segmented regions are obtained. The properties for each region are quantified in step S23, which includes determining a size, shape, morphology, position, orientation or the like of the segmented regions.

In step S24 of FIG. 9, it is determined whether the results from step S23 should be filtered. If so, in step S25, a subset of relevant regions is selected. Such relevant regions can include regions touching an image border, potentially missegmented regions and the like. After step S25 or if no filtering is required (S24), in step S26, the particle 3 is identified and the region is quantified. In step S27, a data table with the results is generated and stored. The data table for example includes one row per region or particle and one column per feature (including the region ID). The data generated and stored in step S27 is for modelling the chemical substance 3. In step S28, which can be performed as an alternative to step S27 or in addition thereto, the segmented results are provided and output as an image for visual presentation and/or validation of the results.

FIG. 10 shows an example of a machine learning model in particular a Convolutional Neural Network (CNN) for image segmentation. Depicted as 100 is an input image. The convolutional neural network in this example comprises an input layer not shown, following the input a layer a filter, also describe as kernel 110, which comprises a kernel, which as a reduced matrix sliding across the input layer, thereby scanning the input image. Following the convolution featured maps 120 are generated. Convolutional layers convolve the input and pass its result to the next layer. In this example, the CNN further comprises one or more pooling layers 120. Pooling layers reduce the dimensions of data by combining the outputs of neuron clusters at one layer into a single neuron in the next layer. In this example this is repeated in convolution layers 140 and pooling layer 150, even further reducing the dimensionality. The CNN may further comprise a flatten layer 140, followed by a fully connected neural network 170 for classifying segments of the image, and provided to output 180. Weights and biases of the CNN may be trained to obtain a candidate segmentation model. During training the structure of the CNN remains unchanged.

Although the present invention has been described in accordance with preferred embodiments, it is obvious for the person skilled in the art that modifications are possible in all embodiments.

### REFERENCE SIGNS

1 input data
2 at least partly labeled images
3 chemical substance
4 labeled chemical substance
5 predicted chemical substance
6 overlap
7 union
8 partly unlabeled data
9 trained machine learning algorithm
10 fully labeled data
11 label data
12 segmentation device
13 storage unit
14 input unit
15 processor
16 output unit
17 connection cable
18 first example
19 second example
20 image
S1 receiving unlabeled/partly labeled image data
S2 receiving machine learning framework (ML) with hyperparameters
S3 training ML framework to obtain a candidate ML algorithm
S4 assessing quality of trained ML algorithm based on validation metric
S5 comparing validation metric with threshold
S6 altering hyperparameters/providing alternative candidate ML framework/algorithm
S7 storing final/selected ML algorithm
S8 receiving (partially) labeled image data
S9 inputting image data to final/selected trained ML algorithm
S10 outputting label data
S11 performing image analysis/determining features
S12 providing performance data
S13 providing receiving (party) labeled image data
S14 providing trained performance model
S15 outputting performance data/parameters
S16 receiving process information/conditions
S17 providing trained particle prediction model
S18 outputting processing conditions
S20 determining postprocessing requirements
S21 performing postprocessing on segmentation label data
S22 obtaining segmented regions
S23 quantifying properties of segmented regions
S24 determining if filter is to be applied
S25 selecting subset of segmented regions
S26 identifying particle and quantifying region
S27 generating and storing data table
S28 outputting segmentation results

1. A training method for training a segmentation model to perform image segmentation on an image of a chemical substance, the training method comprising:
receiving input data including at least partly labeled images comprising one or more partly labeled images of the chemical substance, wherein the label comprises a shape, in particular a set of pixels associated a chemical substance in the image and a position of the chemical substance on the at least partly labeled images;
receiving a machine learning model, in particular comprising a convolutional neural network, having a corresponding set of parameters associated with a structure of the machine learning model;
training the machine learning algorithm model using the input data to obtain a candidate segmentation model for outputting a prediction indicating a shape and position of the chemical substance on input images received as an input, while maintaining the provided set of parameters; and
calculating a validation metric for the candidate machine learning algorithm, the validation metric including an intersection over union per instance, the IoU per instance being a ratio of an overlapping area to a union per instance, the overlapping area being the largest area of overlap between a labeled chemical substance from one of the at least partly labeled images and the prediction by the candidate machine learning algorithm on a corresponding input image corresponding to the one of the at least partly labeled images, and the union being a union of the labeled chemical substance from the one of the at least partly labeled images and the prediction by the candidate machine learning algorithm on the corresponding input image.

2. The training method according to claim 1, further comprising, based on the value of the calculated validation metric, in a new iteration:
providing a set of parameters associated with a structure of the segmentation model, different from the set of parameters initially provided, thereby amending the structure of the segmentation model; and
repeating the steps of receiving the machine learning model, training the machine learning model and calculating the validation metric for the different set of parameters associated with a structure of the machine learning model.

3. The training method according to claim 1, further comprising:
storing and/or outputting a current candidate machine learning algorithm as a trained machine learning algorithm for performing image segmentation if the calculated validation metric is determined as being greater than or equal to a predetermined validation threshold; and/or
storing and/or outputting the candidate machine learning algorithm with the highest validation metric amongst candidate machine learning algorithms from multiple iterations.

4. The training method according to claim 1, wherein the segmentation model is configured to take an image, perform a learned transformation of the image, wherein performing the learned transformation refers to segmentation, and output a list of shapes in the image; wherein the list of shapes refers to shapes identified in the image wherein the machine learning algorithm has a free parameter, in particular a weight that is optimized by heuristic optimization during the training.

5. The training method according to claim 1, wherein the machine learning model is one of the following machine learning models: U-Net or Mask-RCNN (region based convolutional neural network).

6. The training method according to claim 1, wherein the chemical substance is a particle made of a cathode active material, nickel, cobalt and/or manganese.

7. The training method according to claim 1, wherein the at least partly labeled images of the chemical substance are scanning electron microscope (SEM) images.

8. The training method according to claim 1, further including calculating IoU scores using multiple values of an IoU metric and determining a selected value of the IoU metric, the selected value of the IoU metric being the value out of the multiple values of the IoU metric leading to the highest IoU score.

9. The training method according to claim 8, wherein the validation metric corresponds to the IoU per instance score calculated with the selected value of the IoU metric.

10. A segmentation method for performing segmentation of data representing a chemical substance using a trained machine learning algorithm trained according to the training method of claim 1, the segmentation method including:
receiving at least partially unlabeled data to be segmented, the at least partially unlabeled data including an image of the chemical substance;
inputting the at least partially unlabeled data into the trained machine learning algorithm; and
outputting, by the trained machine learning algorithm, label data indicating a shape and position of the chemical substance on the image of the chemical substance.

11. The segmentation method according to claim 10, further including:
using the label data, performing an image analysis to determine features of the represented chemical substance.

12. The segmentation method of claim 11, further comprising
determining a technical performance parameter value of the chemical substance using a performance model, wherein the performance model is parametrized based on technical performance values and features of the chemical substance and using the determined features of the chemical substance as an input to the performance model
providing the technical performance property.

13. The segmentation method according to claim 12, wherein
the performance model is a machine learning model trained using performance training data including features of chemical substances and corresponding performances, the trained performance model being configured to take the features of the represented chemical substance determined through image analysis as an input and to provide performance parameter values as an output.

14. A computer program product comprising instructions which, when the program is executed by a computer, cause the computer to carry out the method according to claim 1.

15. A segmentation device, comprising:
a storage unit for storing a trained machine learning algorithm trained according to claim 1;
an input unit for receiving at least partially unlabeled data to be segmented, the at least partially unlabeled data including an image of the chemical substance;
a processor configured to input the at least partially unlabeled data into the trained machine learning algorithm to determine label data indicating a shape and position of the chemical substance on the image of the chemical substance; and
an output unit for outputting the determined label data.
